(19) United States
(12) Patent Application Publication
(10) Pub. No.: US 2018/0056307 A1
(43) Pub. Date: Mar. 1, 2018

(54) CYCLONE SEPARATOR ARRANGEMENT AND METHOD

(71) Applicant: Valmet AB, Sundsvall (SE)

(72) Inventors: Patrik Pettersson, Alnö (SE); Johan Lindberg, Sundsvall (SE)

(73) Assignee: Valmet AB, Sundsvall (SE)

(21) Appl. No.: 15/556,015

(22) PCT Filed: Feb. 15, 2016
(86) PCT No.: PCT/SE2016/050115

§ 371 Date: Sep. 6, 2017

(30) Foreign Application Priority Data
Mar. 12, 2015 (SE) 1550297-4

(51) Int. Cl. B04C 5/04; C12M 1/00

(52) U.S. Cl. CPC B04C 5/04; C12M 43/00

(57) ABSTRACT

A cyclone separator (10) comprises a pressure chamber (20), an inlet (30) for an incoming flow of a mixture of gas and particles, a gas outlet (50) for outgoing gas arranged through a top wall (26) of the pressure chamber and a particle outlet (40) for outgoing particles arranged in a lower part (22) of the pressure chamber. The pressure chamber has a main rotation symmetric shape. The inlet is arranged through a side wall (28) of an upper part (24) of the pressure chamber for directing the incoming flow with a main velocity component in a tangential direction. The inlet comprises an inlet tube (36) protruding through the side wall of the upper part into the pressure chamber, whereby an inner end (38) of the inlet tube is provided at a position interior of the pressure chamber. A method for operating a cyclone separator is also disclosed.

### TECHNICAL FIELD

The present invention relates in general to methods and arrangement for separating particles from a stream of gas, and in particular to cyclone separator arrangements and methods.

### BACKGROUND

Cyclonic separation used for separating particles from a gas or liquid stream is utilized in many different applications, such as sawmills, oil refineries or when processing biomaterial. In a bioreactor for producing prehydrolyzed particles from plant material, a stream of hot gas comprising particles of prehydrolyzed biomaterial is produced. In order to separate the particles from the hot gas, a cyclone separator is typically utilized.

A problem by using cyclonic separation is that sharp particles may erode the inside of the cyclone separator chamber side wall. In the application of biomaterial processing, it is quite common to have e.g. sand particles mixed with the plant material. Several approaches, having reinforced surfaces of the cyclone separator chamber side wall, have been proposed, but such special treatments are typically expensive to provide and do not improve the situation in a decisive manner.

Another proposed solution within prior art is to provide an additional, particle-free, gas stream at or just before the side wall sections exposed for erosion. This additional gas tends to prohibit the original gas stream to reach the side wall and the erosion is thereby reduced. However, for gas streams comprising relatively large amounts of particles, such as in typical biomaterial applications, the amount of additional gas that is required for mitigating the erosion is large. Both the additional arrangements and the gas that is bled into the cyclone separator will involve increased costs and complexity.

The amount of erosion is strongly dependent on the velocity, with which the particles hit the cyclone chamber side wall. One idea for reducing the erosion is then to reduce the velocity of the gas streaming into the cyclone separator. In the published international patent application WO 02/18056, a cyclone separator inlet nozzle is disclosed, which reduces the inlet speed of the gas stream entering into the cyclone separator. However, a lower entrance speed of the gas reduces the efficiency of the cyclone separation. In a crude approximation, the separation efficiency varies with the square of the tangential velocity, which means that with a lower tangential velocity, the cyclone separation has to operate for a longer time to achieve the same effect. This may to a part be compensated by reduce the mean streaming speed along the symmetry axis of the cyclone separator, thereby increasing the time the gas spends in the cyclone separator. If the entrance velocity is reduced even further, the entire cyclone whirl may even disappear completely and all cyclone separation vanish.

In the Chinese patent application publication CN101773878 A, a cyclone is disclosed, which has an entrance zone with a tangential inlet, a constriction body shrinking upwards arranged above the entrance zone and a cone arranged below the entrance zone. The tangential inlet is configured for increasing the speed of the entering flow.

In the abstract and drawings of the published Korean patent application KR20140056813A, a cyclone separator is disclosed. The cyclone separator comprises an inlet which is formed in a side of the upper part of the cyclone separator, a scroll part of the cyclone separator where the outlet of gas is supplied and a supporting part which is formed between a side of the inside of the inlet and the scroll part. This arrangement prohibits erosion of the scroll part.

Prior art approaches for limiting erosion of the cyclone separator side walls while maintaining a reasonable separation effect still have to be improved.

### SUMMARY

A general object of the present technology is to provide arrangements and methods allowing for reducing erosion of the cyclone separator side walls while maintaining a satisfactory separation effect. The above object is achieved by devices and methods according to the independent claims. Preferred embodiments are defined in dependent claims.

In general words, in a first aspect, a cyclone separator comprises a pressure chamber, an inlet for an incoming flow of a mixture of gas and particles, a gas outlet for outgoing gas arranged through a top wall of the pressure chamber and a particle outlet for outgoing particles arranged in a lower part of the pressure chamber. The pressure chamber has a main rotation symmetric shape. The inlet is arranged through a side wall of an upper part of the pressure chamber for directing the incoming flow with a main velocity component in a tangential direction with respect to the rotation symmetric shape. The inlet comprises an inlet tube protruding through, in the tangential direction, the side wall of the upper part of the pressure chamber into the pressure chamber, whereby an inner end of the inlet tube is provided at a position interior of the pressure chamber.

In a second aspect, a method for operating a cyclone separator comprises introducing of an incoming flow of a mixture of gas and particles into a pressure chamber having a main rotation symmetric shape. The incoming flow has a main velocity component in a tangential direction with respect to the rotation symmetric shape. The introduction of an incoming flow is performed in the tangential direction at a position interior of the pressure chamber. Gas is exited through a gas outlet of the pressure chamber and particles are exited through a particle outlet of the pressure chamber.

One advantage with the proposed technology is that a cyclone separation operation can be obtained with lower velocities of the incoming flow of the mixture of gas and particles. Other advantages will be appreciated when reading the detailed description.

### BRIEF DESCRIPTION OF THE DRAWINGS

The invention, together with further objects and advantages thereof, may best be understood by making reference to the following description taken together with the accompanying drawings, in which:

FIG. 1 is an illustration of an example of a lignocellulosic biomass material treatment arrangement;

FIG. 2A illustrates schematically a prior art cyclone separator in a partial cross-sectional view;

FIG. 2B schematically illustrates a horizontal cross-sectional view of the cyclone separator of FIG. 2A;

FIG. 2C illustrates the equipment of FIG. 2B with a low velocity entering gas flow;

FIG. 3A illustrates an embodiment of a cyclone separator in a partial cross-sectional view;

FIG. 3B illustrates schematically a horizontal cross-sectional view of the embodiment of a cyclone separator of FIG. 3A;

FIGS. 4A-E illustrate schematic cross-sectional views of other embodiments of a cyclone separator;

FIGS. 5A-D illustrate schematically embodiments of a diffuser 60 that can be utilized together with a cyclone separator;

FIGS. 6A-C schematically illustrate embodiments of diffuser arrangements;

FIGS. 7A-D as schematic vertical cross-sections along the flow direction of different embodiments of diffusers;

FIGS. 8A-C illustrate schematically embodiments of inlet systems of a cyclone separator; and

FIG. 9 illustrates a flow diagram of steps of an embodiment of a method for operating a cyclone separator.

### DETAILED DESCRIPTION

Throughout the drawings, the same reference numbers are used for similar or corresponding elements.

For a better understanding of the proposed technology, it may be useful to begin with a brief overview of a system in which the proposed technology can be utilized.

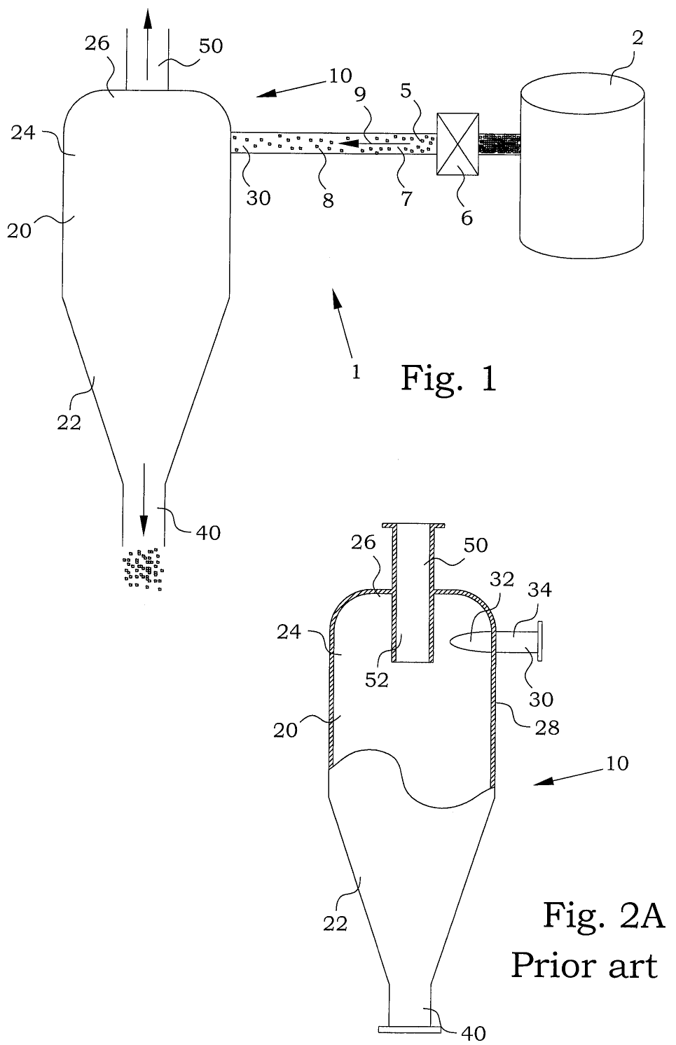

FIG. 1 illustrates a lignocellulosic biomass material treatment arrangement 1, comprising a bioreactor 2, in which biomass material such as wood chips, herbaceous plants, straw, bagasse etc. are treated under high pressure and high temperature to result in pre-hydrolyzed biomass material. The pre-hydrolysis prepares the biomass material for any following hydrolysis step in connection with e.g. fermentation of the biomass. Such pre-hydrolyzed biomass material exits the bioreactor 2 through a transport pipe 5 to a cyclone separator 10. A blow washer arrangement 6 crushes the pre-hydrolyzed biomass material into biomass particles 8, which are transported to the cyclone separator 10 in a flow 9 of a mixture 7 of wet and hot gas and the particles 8, typically also mixed with different types of polluting particles, such as sand. The main fraction of biomass particles may in a typical case be of a size from some tenths of a millimeter up to a couple of millimeters. However, aggregates of particles may be even larger. The transport is performed with a high velocity for mitigate any deposition of biomass particles on the inside of the transport pipe 5. The transport may in a typical case be performed with a steam pressure of 10-15 bar, giving rise to velocities of the gas of some hundred meters per second. The particles and aggregates of particles are carried with the gas and are typically finally reaching velocities in the same order of magnitude.

The flow 9 of the mixture 7 of gas and particles 8 enters into a pressure chamber 20 of the cyclone separator 10 through an inlet 30 for an incoming flow in the upper part 24 of the pressure chamber 20. In the cyclone separator 10, the cyclone action is used to separates out the particles, which are removed from the cyclone separator 10 by a particle outlet 40 for outgoing particles arranged in a lower part 22 of the pressure chamber 20. The remaining cleaned gas exits through a gas outlet 50 for outgoing gas arranged through a top wall 26 of the pressure chamber 20.

When analyzing the prior art approaches for erosion reduction, the approach of utilizing lower velocities than normally applied in the cyclone separator was very attractive, except for the difficulties to maintain the cyclone action inside the cyclone separator. However, in the here presented technology, means for approving the whirl motion are provided, which makes it possible to adapt the general idea of using cyclone separation performed with lower entrance velocities than normally applied.

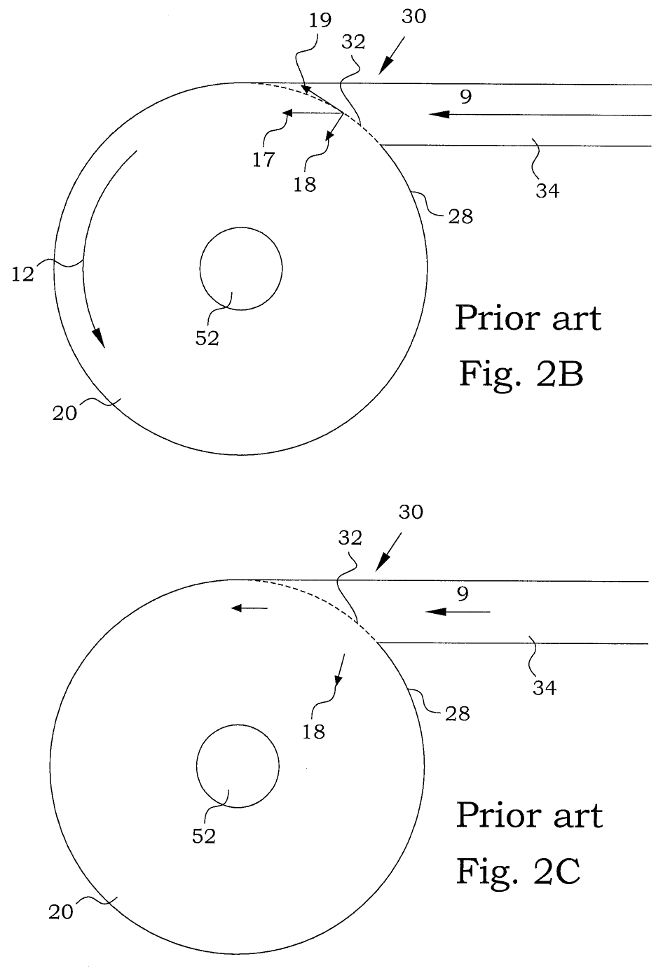

In order to understand the conditions within the cyclone separator, a general description of a prior-art cyclone separator is first given. FIG. 2A illustrates a cyclone separator 10 in a partial cross-sectional view. The pressure chamber 20 has an upper part 24, a lower part 22 and a top wall 26. The inlet 30 is typically provided as an outer pipe 34 attached around a hole 32 in a side wall 28 of the upper part 24 of the pressure chamber 20. The hole 32 is typically provided offset from a center line in order to provide the inflowing gas mixture with a velocity directed mainly in the tangential direction, i.e. the inflowing gas mixture has a significant tangential velocity component. By interaction with the walls of the pressure chamber 20, this tangential velocity component gives rise to a whirl of gas mixture within the pressure chamber 20. The generally heavier and denser particles, compared to the other components in the gas mixture, move in average outwards in the whirl, towards the walls of the pressure chamber 20. The generally less dense gas or steam presents instead a net motion inwards within the whirl towards the center, thus performing a separation of the particles and the gas, respectively, generally referred to as a cyclone action.

The smaller the particles are in the whirl, the less is the separation efficiency by the cyclone action. To that end, in this example, and which is typical for many cyclone separators, the lower part 22 of the pressure chamber 20 has a shape as a frustum of a cone, in order to sharpen up the cyclone action closer to the bottom for separating as fine particles as possible. The particle outlet 40 for outgoing particles is provided at the bottom of the pressure chamber 20 in a lower part 22 of the pressure chamber 20. The gas outlet 50 for outgoing gas is arranged through a top wall 26 of the pressure chamber 20 and comprises an outlet tube 52 protruding downwards from the top wall 26. The outlet tube 52 collects the cleaned gas that is intended to exit the cyclone separator 10.

FIG. 2B schematically illustrates a horizontal cross-sectional view comprising the inlet 30. Here, it is easily seen that the outer pipe 34 is attached to the hole 32 in the side wall 28 displaced from a center direction. This results in that a velocity 17 at the entrance has a significant tangential velocity component 19, which causes the whirl 12 within the pressure chamber 20. The radial velocity component 18 is typically relatively small, however, not totally neglectable. For high velocities, the creation of the whirl 12 will assist in maintaining the whirl 12 despite the radial velocity component 18.

The radial velocity component 18 differs across the hole 32. At the upper edge, as illustrated, of the hole 32, the radial velocity component 18 is neglectable, however, at the lower edge, as illustrated, of the hole 32, the radial velocity component 18 may be very significant, at least for holes 32 that have a diameter that is non-neglectable compared to the diameter of the pressure chamber 20.

FIG. 2C illustrates a similar equipment, but where the entering gas flow has a lower velocity than in earlier examples. Here the velocity in the whirl 12 becomes less, and when the whirl has travelled around the pressure chamber 20 one full turn, the force in the whirl is not enough for dampening the radial velocity component 18. The incoming flow therefore has a tendency to spread out and a part stream can even flow on the opposite side of the outlet tube 52 than intended. The whirl action may even be lost completely.

A basic idea of the present invention is to use the velocity of the incoming flow to more efficiently create the whirl. By introducing the incoming flow in an essentially pure tangential direction and at a position in the interior of the pressure chamber, a whirl can be maintained by much slower inlet gas flows.

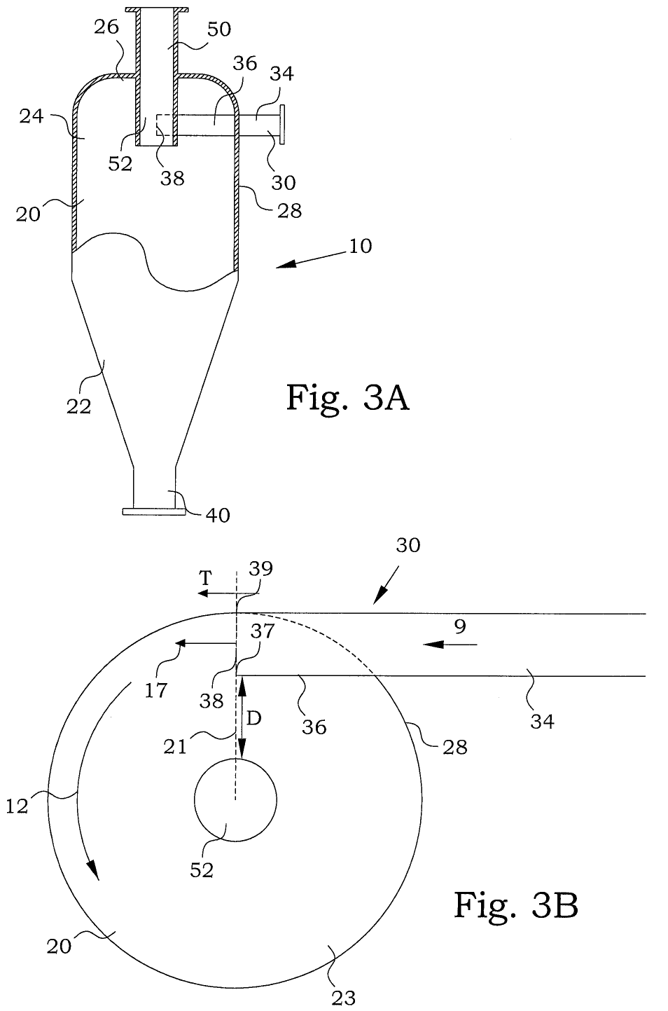

FIG. 3A illustrates an embodiment of a cyclone separator 10 in a partial cross-sectional view. The cyclone separator 10 comprises a pressure chamber 20. The pressure chamber has a main rotation symmetric shape. The cyclone separator 10 further comprises an inlet 30 for an incoming flow 9 of a mixture of gas and particles 8. The inlet 30 is arranged through a side wall 28 of an upper part 24 of the pressure chamber 20. This arrangement is intended for directing the incoming flow 9 with a main velocity component 17 in a tangential direction T with respect to the rotation symmetric shape. The gas outlet 50 and the particle outlet 40 are arranged essentially in the same manner as described above. The inlet 30 here comprises an inlet tube 36 with a constant cross-sectional area protruding through the side wall 28 of the upper part 24 of the pressure chamber 20 into the pressure chamber 20. This protrusion takes place in a tangential direction, as will be discussed in further detail further below. An inner end 38 of the inlet tube 36 is provided at a position interior of the pressure chamber 20. Furthermore, due to the upper and lower walls of the inlet tube 36, flows in the vertical direction is made more difficult.

In a particular embodiment, where the cyclone separator is utilized in a biomass treatment arrangement, an outer end of the inlet tube 36 is connected to an outlet from a bioreactor, as described further above.

FIG. 3B schematically illustrates a horizontal cross-sectional view comprising the inlet 30 of the embodiment of FIG. 3A. Here, it can be seen that the inlet tube 36 protrudes into the interior 23 of the pressure chamber 20. The inner end 38 is in this embodiment positioned at a center line 21. The center line 21 is a line that is perpendicular to the tangential direction and that passes through a center of the pressure chamber 20. The velocity 17 at the entrance into the pressure chamber 20 has then a pure tangential direction. This is true regardless if the gas exists in the top or bottom part, as illustrated, of the inlet tube. Furthermore, since the inner end 38 at least to a part has passed the outlet tube 52, any flow in an opposite whirl direction becomes very unlikely. The whirl movement within the pressure chamber 20 can thereby be maintained by means of lower velocities of the entering gas flows.

In the embodiment of FIG. 3B, the gas outlet comprises an outlet tube 52 protruding downwards from the top wall. A, with respect to the rotation symmetric shape, radially inner edge 37 of the inner end 38 of the inlet tube 36 is provided at a distance D from the outlet tube 52. This preferred detail enables the gas to pass at the radial inner side of the inlet tube 36 when having rotated one or several turns within the pressure chamber 20.

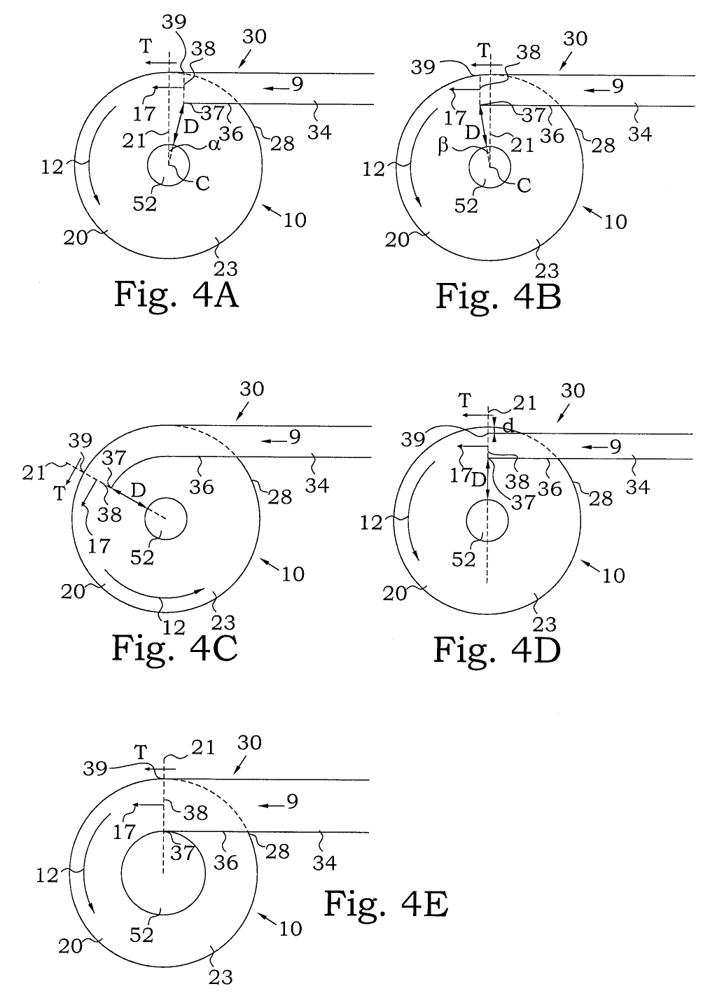

FIG. 4A illustrates a schematic cross-sectional view of another embodiment of a cyclone separator 10. Also here, the inlet tube 36 protrudes through the side wall 28 of the upper part of the pressure chamber 20 into the pressure chamber. The inner end 38 of the inlet tube 36 is provided at a position interior 23 of the pressure chamber 20, however, in this embodiment, the inner end 38 does not reach the entire way to the center line 21. This means that the gas exiting the inner end 38 of the inlet tube 36 at the radially inner side still has a small radial velocity component. However, since the distance to the center line 21 is small, this radial velocity component is also small. It is preferred if an angle a between a line between the center C of the pressure chamber 20 and the radially inner edge 37 of the inner end 38 of the inlet tube 36 and the center line 21 is kept below 30°, which ensures that no part of the gas flow enters the pressure chamber 20 with a radial to tangential velocity component ration that is larger than 1/\sqrt{3}. The closer to the center line 21 the inner end 38 is situated, the smaller becomes the radial velocity component. Therefore, more preferably, the angle α is kept below 20°, even more preferably below 10°, even more preferably below 5° and most preferably in the vicinity or at 0°.

FIG. 4B illustrates a schematic cross-sectional view of another embodiment of a cyclone separator 10. Also here, the inlet tube 36 protrudes through the side wall 28 of the upper part of the pressure chamber 20 into the pressure chamber. The inner end 38 of the inlet tube 36 is provided at a position interior 23 of the pressure chamber 20, however, in this embodiment, the inner end 38 protrudes beyond the center line 21. This means that the gas exiting the inner end 38 of the inlet tube 36 at the radially inner side has a small radial velocity component, in this case directed outwards. A radially velocity component directed outwards will not counteract the whirl formation. However, an outwards directed radially velocity component may increase the erosion on the inner side wall of the pressure chamber 20. It is therefore also preferred to keep such a radial velocity component small. In analogy with the reasoning above, it is preferred if the angle β between the tine between the center C of the pressure chamber 20 and the radially inner edge 37 of the inner end 38 of the inlet tube 36 and the center line 21 is kept below 30°, which ensures that no part of the gas flow enters the pressure chamber 20 with a radial to tangential velocity component ration that is larger than 1/\sqrt{3}. The closer to the center line 21 the inner end 38 is situated, the smaller becomes the outwards directed radial velocity component. Therefore, more preferably, the angle β is kept below 20°, even more preferably below 10°, even more preferably below 5° and most preferably in the vicinity or at 0°.

Considering both FIG. 4A and FIG. 4B, it is thus preferred if an absolute measure of an angle α, β between a line between the center C of the pressure chamber 20 and the radially inner edge 37 of the inner end 38 of the inlet tube 36 and the center line 21, where the center line 21 is a line that is perpendicular to the tangential direction and that passes through the center of the pressure chamber 20, is kept below 30°, more preferably below 20°, even more preferably below 10°, even more preferably below 5° and most preferably in the vicinity or at 0°.

In other words, the action of introducing the incoming flow is performed at a position, for which an absolute measure of an angle α, β between the line between the center C of the pressure chamber 20 and the position and a center line 21, where the center line 21 is a line that is perpendicular to the tangential direction T and passes through the center C of the pressure chamber 20, is smaller than 30°, preferably smaller than 20°, even more preferably smaller than 10°, and even more preferably smaller than 5°.

The most preferred embodiment is as anyone skilled in the art realizes if the inner end 38 protrudes at least up to the center line 21, and most preferably not beyond, as illustrated e.g. in FIG. 3B.

FIG. 4C illustrates a schematic cross-sectional view of yet another embodiment of a cyclone separator 10. Also here, the inlet tube 36 protrudes through the side wall 28 of the upper part of the pressure chamber 20 into the pressure chamber. The inner end 38 of the inlet tube 36 is provided at a position interior 23 of the pressure chamber 20, and, in this embodiment, the inner end 38 is also positioned at the center line 21. In this embodiment, however, the inlet tube 36 is curved to follow the wall of the pressure chamber 20. The center line 21 is directed perpendicular to the tangential direction as defined at the inner end 38. This embodiment also provides a good possibility to achieve a whirl action within the pressure chamber 20 by relatively low entrance velocities of the gas. A small disadvantage is, however, that the curved shape of the inlet tube 36 increase the complexity in manufacturing.

In the embodiments presented here above, a, with respect to the rotation symmetric shape, radially outer edge 39 of the inner end 38 of the inlet tube 36 is provided against the side wall 28 of the upper part of the pressure chamber 20 or is integrated with the side wall 28 of the upper part of the pressure chamber 20. Typically, such arrangement will utilize the space within the pressure chamber 20 in an optimal way. However, as illustrated in the embodiment of FIG. 4D, in manufacturing of the cyclone separator 10, it might in certain cases be easier to provide the radially outer edge 39 at a distance d from the side wall 28 of the pressure chamber 20. The distance d may in different embodiments be within 0 and 25% of the diameter of the pressure chamber 20, but is preferably kept small. Thus, preferably, the distance d is within 0 and 15%, more preferably within 0 and 5% and most preferably within 0 and 2.5% of the diameter of the pressure chamber 20 of the pressure chamber 20. The efficiency of creating the whirl within the pressure chamber 20 will typically be reduced, however, typically marginally, and this marginal loss in efficiency may instead be compensated by the ease of manufacture.

In FIG. 4E, an embodiment of a cyclone separator 10 is illustrated, having an outlet tube 52 protruding downwards from the top wall, which outlet tube 52 is wider than in previous embodiments. If also the inlet tube 36 is wider than in previous embodiments, the inner end 38 of the inlet tube 36 may be provided against the outlet tube 52. In other words, the, with respect to the rotation symmetric shape, radially inner edge of the inner end 38 of the inlet tube 36 is provided without any distance from the outlet tube 52. This, non-preferred embodiment, may be operable in some applications, in particular where non-sticky particles are to be separated. However, it is believed that in most cases, there might be problems with solid matter that collects at the outside of the inlet tub 36.

As discussed further above, the here presented technology is very useful in connection with incoming flows of a mixture of gas and particles that have relatively low velocities, compared to prior art cyclones, in order to reduce the erosion of the cyclone chamber. However, in a typical case, where a mixture of gas and particles is to be moved over a distance between e.g. a pre-hydrolyzer and a cyclone chamber, there is no general request to have a low velocity of such a flow. At the contrary, low velocities when transporting flows of a mixture of gas and particles may render into deposition of solid particles at the inner walls of the transporting tubes. Therefore, high velocities are typically utilized during transporting of flows of a mixture of gas and particles, as mentioned above typically in the order of a couple of hundred meters per second. In the embodiments of FIGS. 3A-B and 4A-E, an inlet tube having a constant cross-sectional area has been illustrated, which basically means that the velocities of the particles are kept constant during the flow through the inlet tube.

It is therefore common that there has to be a reduction in velocity of such a flow before letting the flow into a cyclone separator, in order to reduce the erosion of the inner side walls of the cyclone chamber. Therefore, in a preferred embodiment, the inlet comprises a diffuser. The diffuser is an arrangement that distributes a flow over an increased area, which results in a decreased average velocity. In other words, the inlet tube is provided with an increasing cross-sectional area.

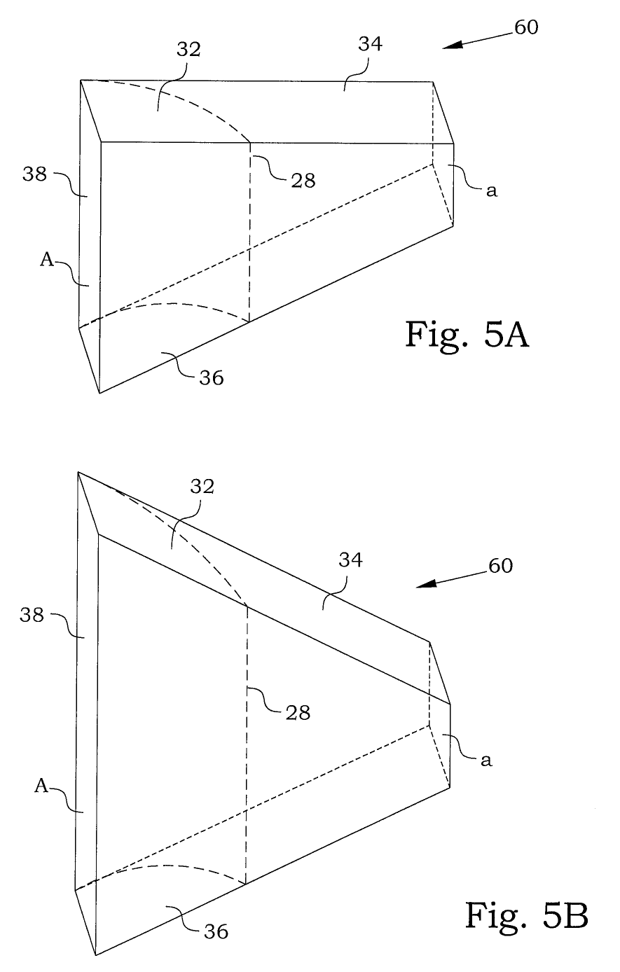

FIG. 5A illustrates schematically an embodiment of a diffuser 60 that can be utilized together with any of the above presented embodiments of a cyclone separator. In this embodiment, the diffuser 60 comprises a part of the inlet tube 36. In this embodiment, the diffuser 60 also comprises a part of the outer pipe 34 of the inlet. The diffuser 60 has a monotonically increasing cross-sectional area towards the inner end 38 of the inlet tube 36. The term “monotonically increasing cross-sectional area” is intended to define a cross-sectional area that is non-decreasing in all portions, i.e. only has portions of increasing and/or constant cross-sectional areas towards the inner end 38 of the inlet tube 36. Note, however, that the increasing does not necessarily have to be strictly increasing, i.e. there might also be portions with constant cross-sectional areas. An ingoing cross-sectional area a is increased monotonically to an outgoing cross-sectional area A, in this particular embodiment the cross-sectional area is strictly increasing. Preferably, the cross-sectional area of the diffuser 60 increases at least 2 times, more preferably at least 5 times and most preferably around 10 times from the ingoing cross-sectional area a to the outgoing cross-sectional area A. On the other hand, the velocity reduction cannot in practice be too large in order to maintain an efficient cyclone action and it is therefore preferred if the cross-sectional area of the diffuser 60 increases at most 17 times, and more preferably at most 13 times from the ingoing cross-sectional area a to the outgoing cross-sectional area A. The position of the side wall 28 of the pressure chamber and the hole 32 in the pressure chamber are depicted by broken lines, to increase the understanding of the position of the diffuser 60. In the present embodiment, the monotonically increasing cross-sectional area of the diffuser 60 is provided by a monotonically increased vertical dimension of the diffuser 60 towards the inner end 38 of the inlet tube 36. In particular, the vertical dimension of the diffuser 60 is increased downwards, towards the inner end 38 of said inlet tube 36. In other words, the upper wall of the diffuser 60 is essentially horizontal, while the lower wall is sloping downwards.

This increase in cross-sectional area leads to a reduction of the velocity of the incoming flow before introducing the incoming flow into the pressure chamber. In the view of the above discussion, preferably, the velocity reduction is at least 2 times, more preferably at least 5 times and most preferably around 10 times from the ingoing cross-sectional area a to the outgoing cross-sectional area A. On the other hand, the velocity reduction cannot in practice be too large in order to maintain an efficient cyclone action and it is therefore preferred if the velocity reduction is at most 17 times, and more preferably at most 13 times from the ingoing cross-sectional area a to the outgoing cross-sectional area A.

FIG. 5B illustrates schematically another embodiment of a diffuser 60 that can be utilized together with any of the above presented embodiments of a cyclone separator. This embodiment resembles the embodiment of FIG. 5A e.g. in that the monotonically increasing cross-sectional area of the diffuser 60 is provided by a monotonically increased vertical dimension of the diffuser 60 towards the inner end 38 of the inlet tube 36. In this embodiment, however, the upper wall of the diffuser 60 slopes upwards.

FIG. 5C illustrates schematically yet another embodiment of a diffuser 60 that can be utilized together with any of the above presented embodiments of a cyclone separator. This embodiment resembles the earlier embodiments of FIGS. 5A and 5B e.g. in that the diffuser 60 has a monotonically increasing cross-sectional area towards the inner end 38 of the inlet tube 36. In this embodiment, however, the increased cross-sectional area of the diffuser 60 is provided by increasing both the horizontal and vertical dimensions towards the inner end 38.

It is presently believed that the embodiment in FIG. 5A is the preferred embodiment among these alternatives. However, depending on the actual application, also the embodiments of FIGS. 5B and 5C are operable, providing the intended technical effect, giving a reduced velocity upon entering the cyclone separator. The embodiment of FIG. 5C has the drawback that it allows the mixture of gas and particles in the diffuser 60 to achieve a radial velocity component. This radial velocity component is typically small compared to the total velocity, however, since this is an unwanted feature when the mixture enters into the cyclone separator, the increase in the horizontal dimension is preferably kept small compared to the total horizontal dimension at the inner end 38. The embodiment of FIG. 5B allows the mixture of gas and particles in the diffuser 60 to achieve a small vertical velocity component, directed upwards. In a typical cyclone separator, the inlet is provided relatively close to the upper wall, and a velocity component directed upwards will only cause a generally undesired interaction between the top wall of the cyclone separator and the flow of the mixture. Therefore, the presently preferred embodiment has a diffuser with a horizontal upper wall.

The actual shape of the diffuser and/or inlet tube 36 is in general not particularly important. In the previous embodiments, tubes of rectangular cross-sections have been illustrated. This is typically easy to integrate with cyclone separator pressure chambers having a cylindrical form in the upper part. Such rectangular cross-sections can therefore in a practical manufacturing point of view be considered as advantageous. However, in general, also other types of cross-sectional geometries are feasible. FIG. 5D illustrates one example, where the diffuser 60 has an elliptical cross-section.

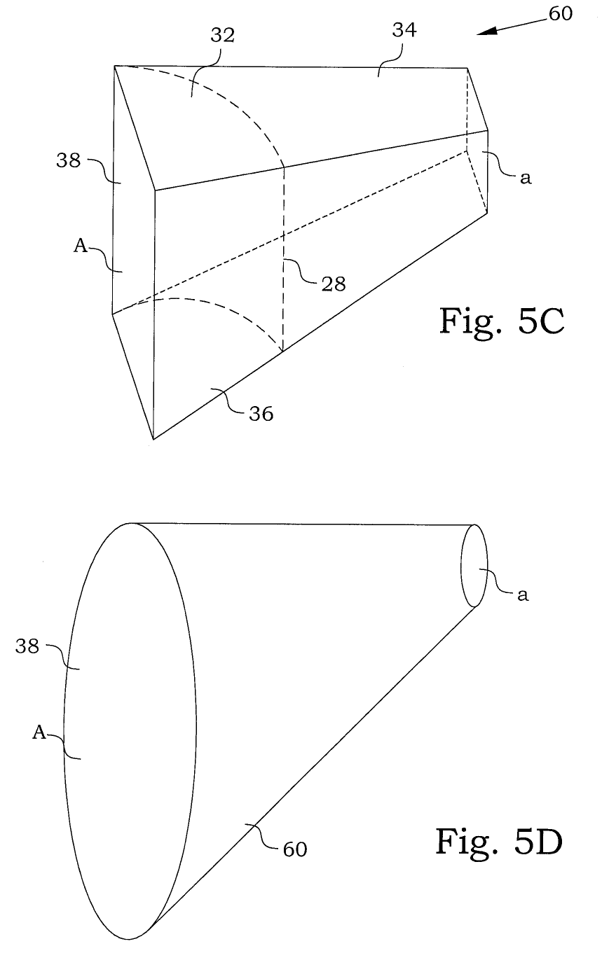

As mentioned above, the diffuser could comprise at least a part of the inlet tube 36 and/or at least a part of the outer pipe 34 of the inlet. FIG. 6A schematically illustrates an embodiment of a diffuser arrangement, where the diffuser 60 has one part outside the side wall 28 and one part inside the side wall 28, i.e. in the interior 23 of the pressure chamber. The part outside the side wall 28 then constitutes a part of the outer pipe 34 of the inlet. The part inside the side wall 28 constitutes a part of the inlet tube 36. FIG. 6B schematically illustrates an embodiment of a diffuser arrangement, where the diffuser 60 is provided entirely inside the side wall 28, i.e. in the interior 23 of the pressure chamber. The diffuser 60 then constitutes at least a part of the inlet tube 36. FIG. 6C schematically illustrates an embodiment of a diffuser arrangement, where the diffuser 60 is provided entirely outside the side wall 28. The diffuser 60 then constitutes at least a part of the outer pipe 34 of the inlet.

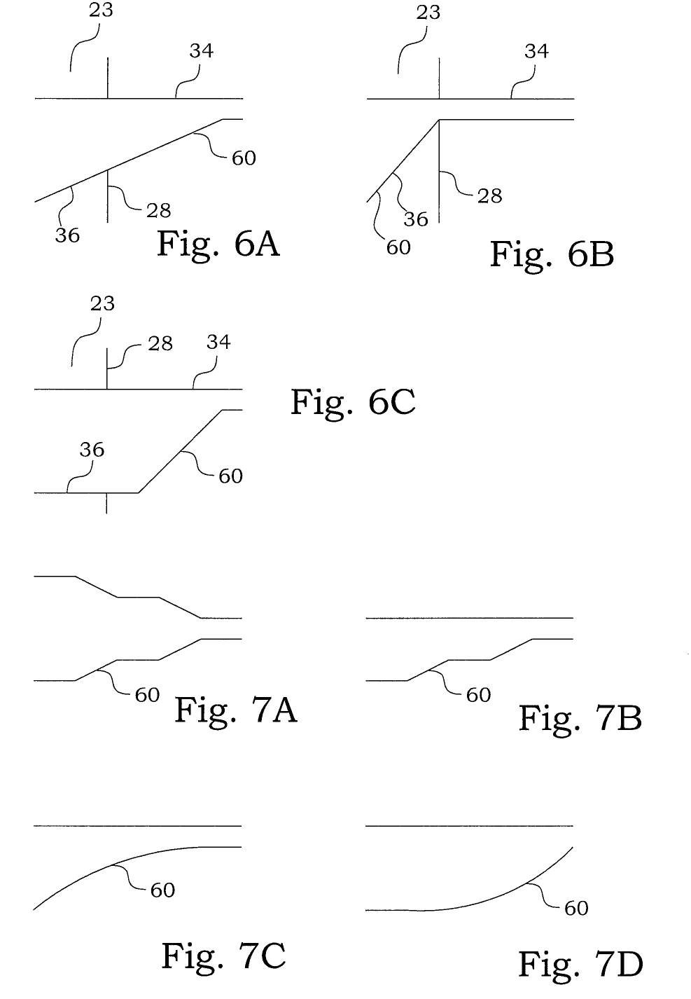

The behavior of the increase of the diffuser cross-sectional area can be designed in different ways. A few non-limiting embodiments are schematically illustrated in the FIGS. 7A-7D. In FIG. 7A, the diffuser cross-sectional area does not increase continuously, i.e. it is not strictly increasing, but increases instead in two separated regions, and thus still presenting a monotonic increase. In this embodiment, the vertical dimension change of the diffuser 60 takes place both upwards and downwards. In this particular embodiment, in the portions where the diffuser cross-sectional area increases, the vertical dimension increases linearly, i.e. in the cross-sectional view, the upper and lower walls of the diffuser have the form of a straight line. In FIG. 7B, a similar embodiment is illustrated. However, in this case, the change in the vertical dimension is provided by the lower wall of the diffuser 60. In FIG. 7C, an embodiment of a diffuser 60 having a non-linear increase of the vertical dimension is illustrated. Also in FIG. 7D, an embodiment of a diffuser 60 having a non-linear increase of the vertical dimension is illustrated. The non-linear increase of the vertical dimension in FIGS. 7C and 7D can be seen as curved lines.

It can be noted that when a flow of a mixture of a gas and particles enters a cyclone separator with a low velocity, the separation efficiency is generally lower compared to a case where a high entrance velocity is used. The cyclone action increase in general with increasing velocity in the whirl. This reduced efficiency may at least to a part be compensated by allowing the gas/particle mixture to spend more time in the cyclone separator, thus travelling more turns around the pressure chamber before they are exiting from the cyclone. One way to do that is to try to reduce the vertical velocity induced when entering of the flow of the mixture from the outer pipe into the pressure chamber. If the mixture is entered with a pure horizontal velocity, the movement downwards is only caused by the gravity, typically on the particles, and the gas pressure from the subsequent entered mixture. The mixture will therefore be kept in a whirl motion as long as possible, which increase the separation efficiency.

Such operation can at least to a part be made more difficult to obtain if the mixture already at the instant of entering the pressure chamber has a downwards directed velocity component. It is therefore concluded that approaches of entering the mixture through the top wall, thereby deliberately giving a large vertical velocity component are basically unsuitable.

Also the provision of a diffuser may, as briefly mentioned above, give a minor vertical velocity component. However, by an appropriate design of the diffuser, such effects can be reduced. One possibility is to provide the actual diffuser action a distance before the inner end of the inlet tube, and providing a constant cross-sectional area part of the inlet tube closest to the inner end. The embodiments of FIGS. 7A, 7B and 7D are examples of such designs.

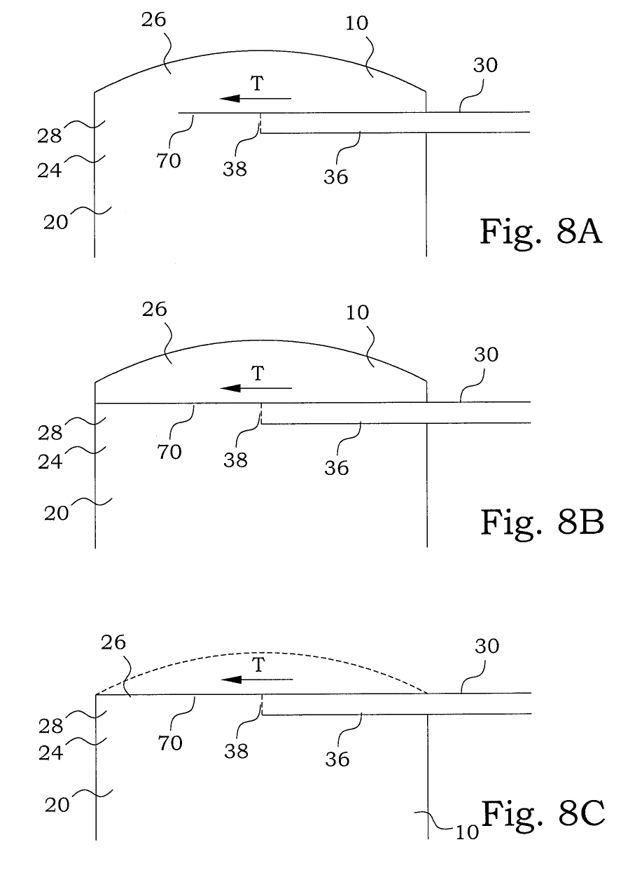

During experiments with cyclone separators operating with low entrance velocities, as compared with typical prior art cyclones, it has been found that there is a tendency for the particles in the mixture entering the pressure chamber to stick to the top wall and/or the side wall above the entering level. This has been particularly frequent for sticky particles. In many applications, remaining quantities of particles in the cyclone pressure chamber are disadvantageous. In FIG. 8A an embodiment of an inlet system of a cyclone separator is illustrated schematically. The inlet tube 36 is here provided with a baffle plate 70. The baffle plate 70 is arranged horizontally and is attached to an upper part of the inner end 38 of the inlet tube 36 and protrudes in the tangential direction T of the incoming mixture of gas and particles. This arrangement of the baffle plate 70 guides the streaming mixture towards to the opposite side wall of the pressure chamber. Any particles having an upwards directed vertical velocity component is thereby prevented to reach the top of the pressure chamber. This arrangement of the baffle plate 70 is advantageously combined with e.g. the diffuser described in other embodiments further above, and may even mitigate the disadvantages of certain embodiments providing upwards directed vertical velocity components, e.g. as could be provided by the embodiment of FIG. 5B.

In FIG. 8B another embodiment of an inlet system of a cyclone separator is illustrated schematically. Here the horizontal baffle plate 70 protrudes in the tangential direction T all the way to the side wall of the pressure chamber 20. Such an arrangement provides for that most of the particles are caught by the whirl motion before they have any opportunity to deviate upwards towards the top.

In certain applications, as e.g. in the embodiment of FIG. 8C, the inlet 30 can be provided juxtaposed to the top wall 26 or a divisional top wall of the pressure chamber 20. The horizontal baffle plate 70 is then preferably integrated in the top wall 26 of the pressure chamber 20.

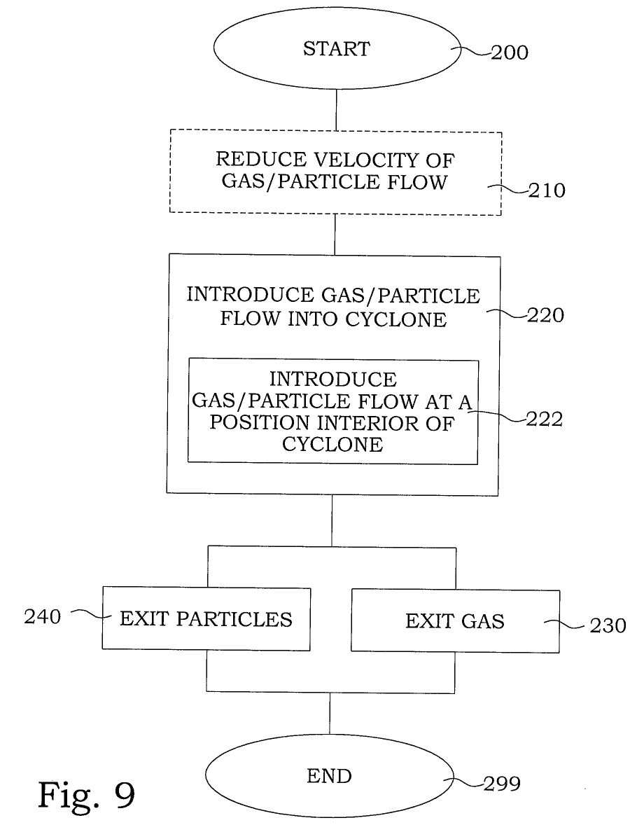

FIG. 9 illustrates a flow diagram of steps of an embodiment of a method for operating a cyclone separator. The procedure starts in step 200. In step 220, an incoming flow of a mixture of gas and particles is introduced into a pressure chamber having a main rotation symmetric shape. The incoming flow has a main velocity component in a tangential direction with respect to the rotation symmetric shape. The step 220 comprises a part step 222 in which the introduction of the incoming flow is performed in the tangential direction at a position interior of the pressure chamber. In a preferred embodiment, the step of introducing an incoming flow is performed, in the tangential direction, at or behind a center line. The center line is defined as a line that is perpendicular to the tangential direction and that passes through a center of the pressure chamber. In step 230, gas is exited through a gas outlet of the pressure chamber and in step 240, particles are exited through a particle outlet of the pressure chamber. The procedure ends in step 299.

In a particular embodiment, the step 220 is performed while maintaining or reducing a velocity of the incoming flow before entering into the pressure chamber. In a preferred embodiment, the method for operating a cyclone separator comprises the further step 210 of reducing a velocity of the incoming flow before the step of introducing the incoming flow into the pressure chamber.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. A cyclone separator, comprising:
a pressure chamber having main rotation symmetric shape;
an inlet for an incoming flow of a mixture of gas and particles, said inlet being arranged for directing said incoming flow with a main velocity component in a tangential direction with respect to said rotation symmetric shape;
a gas outlet for outgoing gas arranged through a top wall of said pressure chamber; and
a particle outlet for outgoing particles arranged in a lower part of said pressure chamber;
wherein said inlet being arranged through a side wall of an upper part of said pressure chamber and comprises an inlet tube protruding through, in said tangential direction, said side wall of said upper part of said pressure chamber into said pressure chamber, whereby an inner end of said inlet tube is provided at a position interior of said pressure chamber.

2. The cyclone separator according to claim 1, wherein an absolute measure of an angle between a line between a center of said pressure chamber and a radially inner edge of said inner end of said inlet tube and a center line, said center line being a line that is perpendicular to said tangential direction and passing through said center of said pressure chamber, is smaller than 30°.

3. The cyclone separator according to claim 2, wherein said inlet tube protrudes into said interior of said pressure chamber up to said center line.

4. The cyclone separator according to claim 1, wherein a, with respect to said rotation symmetric shape, radially outer edge of said inner end of said inlet tube is provided against said side wall of said upper part of said pressure chamber or is integrated with said side wall of said upper part of said pressure chamber.

5. The cyclone separator according to claims 1, wherein
said gas outlet comprises an outlet tube protruding downwards from said top wall; and
wherein a, with respect to said rotation symmetric shape, radially inner edge of said inner end of said inlet tube is provided at a distance from said outlet tube.

6. The cyclone separator according to claim 1, wherein said inlet has a constant or increasing cross-sectional area.

7. The cyclone separator according to claim 6, wherein said inlet comprises a diffuser.

8. The cyclone separator according to claim 7, wherein said diffuser comprises a part having a monotonically increasing cross-sectional area towards said inner end of said inlet tube.

9. The cyclone separator according to claim 8, wherein a cross-sectional area of said diffuser increase at least 2 times from an ingoing cross-sectional area to an outgoing cross-sectional area.

10. The cyclone separator according to claim 8, wherein said monotonically increasing cross-sectional area of said diffuser is provided by a monotonically increased vertical dimension of said diffuser towards said inner end of said inlet tube.

11. The cyclone separator according to claim 10, wherein said vertical dimension of said diffuser is increased downwards, towards said inner end of said inlet tube.

12. The cyclone separator according to claim 1, further comprising a horizontal baffle plate attached to an upper part of said inner end of said inlet tube and protruding in said tangential direction.

13. The cyclone separator according to claim 12, wherein said horizontal baffle plate protrudes in said tangential direction all the way to said side wall of said pressure chamber.

14. The cyclone separator according to claim 13, wherein said horizontal baffle plate is integrated in said top wall of said pressure chamber.

15. The cyclone separator according to claim 1, wherein an outer end of said inlet tube is connected to an outlet from a bioreactor.

16. A method for operating a cyclone separator, comprising the steps of:
introducing an incoming flow of a mixture of gas and particles into a pressure chamber having main rotation symmetric shape, said incoming flow having a main velocity component in a tangential direction with respect to said rotation symmetric shape;
exiting gas through a gas outlet of said pressure chamber; and
exiting particles through a particle outlet of said pressure chamber;
wherein said step of introducing an incoming flow is performed through a side wall of an upper part of said pressure chamber in said tangential direction at a position interior of said pressure chamber.

17. The method according to claim 16, wherein said step of introducing an incoming flow is performed at a position, for which an absolute measure of an angle between a line between a center of said pressure chamber and said position and a center line, said center line being a line that is perpendicular to said tangential direction and passing through said center of said pressure chamber, is smaller than 30°.

18. The method according to claim 17, wherein said step of introducing an incoming flow is performed, in said tangential direction, at said center line.

19. The method according to claim 16, wherein said step of introducing an incoming flow is performed while maintaining or reducing a velocity of said incoming flow before entering into said pressure chamber.

20. The method according to claim 19, comprising the further step of:
reducing said velocity of said incoming flow before said step of introducing said incoming flow into said pressure chamber.
